(19) United States
(12) Patent Application Publication
(10) Pub. No.: US 2004/0023224 A1
(43) Pub. Date: Feb. 5, 2004

(54) Maintaining scale factor in an instrument for reading a biopolymer array

(76) Inventors: John F. Corson, Mountain View, CA (US); Andreas N. Dorsel, Menlo Park, CA (US); Debra A. Sillman, Los Altos, CA (US); Jayati Ghosh, San Jose, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/212,191

(22) Filed: Jul. 31, 2002

(51) Int. Cl. C12Q 1/68; G01N 33/53; G06F 19/00; G01N 33/48; G01N 33/50

(52) U.S. Cl. 435/6; 702/19; 435/7.1

(57) ABSTRACT

A method, apparatus for executing the method, and computer program products for use in such an apparatus. The method includes scanning an interrogating light across multiple sites on an array package including an addressable array of multiple features of different moieties, which scanned sites include multiple array features. Signals from respective scanned sites emitted in response to the interrogating light are detected. In the subject methods, a scanner is employed in which the interrogating light and detector gain are modulated in a manner sufficient to maintain constant scale factor in the scanner despite reductions in laser power resulting from laser degradation.

### FIELD OF THE INVENTION

This invention relates to arrays, particularly biopolymer arrays such as DNA arrays, which are useful in diagnostic, screening, gene expression analysis, and other applications, and particular to biopolymer array optical scanners employed therewith.

### BACKGROUND OF THE INVENTION

Polynucleotide arrays (such as DNA or RNA arrays), are known and are used, for example, as diagnostic or screening tools. Such arrays include features (sometimes referenced as spots or regions) of usually different sequence polynucleotides arranged in a predetermined configuration on a substrate. The array is “addressable” in that different features have different predetermined locations (“addresses”) on a substrate carrying the array.

Biopolymer arrays can be fabricated using in situ synthesis methods or deposition of the previously obtained biopolymers. The in situ synthesis methods include those described in U.S. Pat. No. 5,449,754 for synthesizing peptide arrays, as well as WO 98/41531 and the references cited therein for synthesizing polynucleotides (specifically, DNA). In situ methods also include photolithographic techniques such as described, for example, in WO 91/07087, WO 92/10587, WO 92/10588, and U.S. Pat. No. 5,143,854. The deposition methods basically involve depositing biopolymers at predetermined locations on a substrate, which are suitably activated such that the biopolymers can link thereto. Biopolymers of different sequence may be deposited at different feature locations on the substrate to yield the completed array. Washing or other additional steps may also be used. Procedures known in the art for deposition of polynucleotides, particularly DNA such as whole oligomers or cDNA, are described, for example, in U.S. Pat. No. 5,807,522 (touching drop dispensers to a substrate), and in PCT publications WO 95/25116 and WO 98/41531, and elsewhere (use of an ink jet type head to fire drops onto the substrate).

In array fabrication, the quantities of DNA available for the array are usually very small and expensive. Sample quantities available for testing are usually also very small and it is therefore desirable to simultaneously test the same sample against a large number of different probes on an array. These conditions require the manufacture and use of arrays with large numbers of very small, closely spaced features.

The arrays, when exposed to a sample, will exhibit a binding pattern. The array can be interrogated by observing this binding pattern by, for example, labeling all polynucleotide targets (for example, DNA) in the sample with a suitable label (such as a fluorescent compound), scanning an interrogating light across the array and accurately observing the fluorescent signal from the different features of the array. Assuming that the different sequence polynucleotides were correctly deposited in accordance with the predetermined configuration, then the observed binding pattern will be indicative of the presence and/or concentration of one or more polynucleotide components of the sample. Peptide arrays can be used in a similar manner. Techniques for scanning arrays are described, for example, in U.S. Pat. No. 5,763,870 and U.S. Pat. No. 5,945,679. However, the signals detected from respective features emitted in response to the interrogating light, may be other than fluorescence from a fluorescent label. For example, the signals may be fluorescence polarization, reflectance, or scattering, as described in U.S. Pat. No. 5,721,435.

Instruments for reading biopolymer arrays, i.e., array scanners, typically use a laser as an interrogating light source, which is scanned over the array features. Particularly in array scanners used for DNA sequencing or gene expression studies, a detector (typically a fluorescence detector) with a very high light sensitivity is normally desirable to achieve maximum signal-to-noise in detecting hybridized molecules. At present, photomultiplier tubes (“PMTs”) are still the detector of choice although charge coupled devices (“CCDs”) can also be used. PMTs are typically used for temporally sequential scanning of array features, while CCDs permit scanning many features in parallel.

Laser output power in such array scanners tends to drift over time, e.g., in response to the gradual degradation of the laser. This drift can cause a decrease in the scale factor of the scanner, which is defined as the number of signal counts that are reported to the user per chromophore per area on the array. The scale factor decreases because, as the output power of the laser decreases, the number of signal counts generated per chromophore per area also decreases. Decreases in scale factor are undesirable from a user's standpoint, and should be avoided if possible. It is desirable that the instrument maintain a constant scale factor over time so that experiments performed at different times can be directly compared.

In order to maintain a constant scale factor as the laser degrades over time, one approach that has been employed is to limit the fraction of the laser power that reaches the chromophores on the array surface (i.e., the interrogating power), so that as the laser degrades a larger fraction of the laser power is allowed to reach the array surface and thereby excite the chromophores present thereon. Specifically, laser output modulators, e.g., electro-optic modulators, variable ND filters, acousto-optic modulators, movable shutters, etc., are employed to initially set the laser output power that reaches the array surface to a level below the maximum possible level. This level of laser power passing through the modulator and reaching the array surface/sample is conveniently referred to as the control point. The laser power is then monitored and, as the laser degrades, the modulator allows ever more of the laser's power to pass through, thus holding the power at the sample point (i.e., the interrogating power) constant, and thereby maintaining a constant scale factor despite laser degradation.

While the above approach provides for a constant scale factor despite laser degradation, it does suffer from limitations. For example, below a certain laser power it becomes difficult to successfully control the control loop that runs the modulator because some margin between the maximum total power of the laser and the power control point is required to avoid control loop instability.

As such, once the laser output falls to a level below which stability cannot be controlled, the control point is reset to a lower value to maintain control loop stability. However, in resetting the control point to a lower value, the scale factor is abruptly decreased because the interrogating power reaching the sample on the array surface is decreased. Such an abrupt decrease in interrogating power is not desirable, as it negatively impacts the use of the scanner and results obtained thereby.

As such, there is a continued need for an improved scanning system which provides for a constant scale factor despite a resetting of a control point and concomitant decrease in interrogating power, so that a constant scale factor can be provided for at least two different interrogating powers.

### RELEVANT LITERATURE

Representative optical scanners of interest include those described in U.S. Pat. Nos. 5,585,639; 5,760,951; 5,763,870; 6,084,991; 6,222,664; 6,284,465; 6,329,196; 6,371,370 and 6,406,849.

### SUMMARY OF THE INVENTION

The present invention then, provides an instrument for reading a biopolymer array, i.e., an optical scanner, and method of using an optical scanner with an addressable array of multiple features of different moieties. These moieties may, for example, be polynucleotides (such as DNA or RNA) of different sequences for different features. In the method, an interrogating light is scanned across the array by an optical scanner. This scanning can be accomplished, for example, by moving the interrogating light relative to the array, moving the array relative to the interrogating light, or both. The interrogating light is generated from a variable optical attenuator through which light from a light source has passed, and which optical attenuator is responsive to a control signal to alter the power of the interrogating light. Signals from respective features emitted in response to the interrogating light are then detected by a suitable detector, e.g., a PMT or other light detector element. A feature of the subject methods is that the scanner employed therein is one that is capable of maintaining a constant scale factor during use, despite light source degradation and a decrease in the control point from a first to a second value. The constant scale factor is maintained through modulation of the interrogating power and modulation of the detector gain.

The present invention further provides an apparatus, i.e., a biopolymer array optical scanner, for executing methods of the present invention, i.e., for maintaining a constant scale factor through modulation of detector gain, despite a decrease in interrogating power from a first to a second value. In a first aspect, the apparatus includes the light source and variable optical attenuator, a scanning system to control scanning, an emitted signal detector whose gain can be modulated to provide for the desired constant scale factor, and a power detector to detect the power of the interrogating light. The apparatus also includes a system controller which receives input from, and controls the remainder of, the apparatus as required (including using location information or making determinations, as described above) such that the remainder of the apparatus can execute a method of the invention, i.e., maintain a constant scale factor through modulation of interrogating power and detector gain. For example, the system controller may adjust the optical attenuator control signal to alter interrogating light power, based on the power detected by the power detector until a first control point is reached, following which a second control point is set and a concomitant modulation in detector gain is effected to maintain a constant scale factor.

The present invention further provides a computer program product for use in an apparatus of the present invention. Such a computer program product includes a computer readable storage medium having a computer program stored thereon which, when loaded into a computer of the apparatus, such as the controller, causes it to perform the steps required by the apparatus to execute a method of the present invention, i.e., to maintain a constant scale factor by modulation of interrogating power and detector gain.

While the methods and apparatus have been described in connection with arrays of various moieties, such as polynucleotides or DNA, other moieties can include any chemical moieties such as biopolymers. Also, while the detected signals may particularly be fluorescent emissions in response to the interrogating light, other detected signals in response to the interrogating light can include polarization, reflectance, or scattering, signals.

In addition, the design disclosed in this patent application can be extended to the case of a non-linear relationship between fluorescence signal and control point (or laser power reaching the sample). For example, if calibration data are acquired and stored, these data can be used later to compensate for such non-linear dependencies, e.g., due to saturation of the fluorescent dye label used.

The method, apparatus, and kits of the present invention can provide any one or more of the following or other benefits. Correction in the power of an interrogating light to maintain constant scale factor can be obtained. Increased notice periods prior to laser replacement may also be obtained. Scale factor adjustment can be delayed. In addition, increased laser lifetime can be realized. Furthermore, the subject invention provides yet additional benefits, the above specific benefits being merely representative.

### BRIEF DESCRIPTION OF THE DRAWINGS

Embodiments of the invention will now be described with reference to the drawings, in which:

FIG. 1 is a perspective view of a substrate carrying a typical array, as may be used with, or part of, a package of the present invention;

FIG. 2 is an enlarged view of a portion of FIG. 1 showing some of the identifiable individual regions of a single array of FIG. 1;

FIG. 3 is an enlarged cross-section of a portion of FIG. 2;

FIG. 4 is a front view of an array package in the form of a cartridge;

FIG. 5 illustrates an apparatus of the present invention; and

FIG. 6 is a flowchart illustrating a method of the present invention.

To facilitate understanding, the same reference numerals have been used, where practical, to designate similar elements that are common to the FIGS.

### DETAILED DESCRIPTION OF THE INVENTION

Throughout the present application, unless a contrary intention appears, the following terms refer to the indicated characteristics. A “biopolymer” is a polymer of one or more types of repeating units. Biopolymers are typically found in biological systems and particularly include peptides or polynucleotides, as well as such compounds composed of or containing amino acid or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5-carbon sugar and a nitrogen containing base, as well as analogs (whether synthetic or naturally occurring) of such sub-units. For example, a “biopolymer” includes DNA (including cDNA), RNA, oligonucleotides, and PNA and other oligonucleotides as described in U.S. Pat. No. 5,948,902 and references cited therein (all of which are incorporated herein by reference), regardless of the source. An “oligonucleotide” generally refers to a polynucleotide of about 10 to 100 nucleotides (or other units) in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution). An “addressable array” includes any one, two, or three dimensional arrangement of discrete regions (or “features”) bearing particular moieties (for example, different polynucleotide sequences) associated with that region and positioned at particular predetermined locations on the substrate (each such location being an “address”). An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). These regions may or may not be separated by intervening spaces.

A “processor” references any hardware and/or software combination which will perform the functions required of it. For example, any processor herein may be a programmable digital microprocessor such as available in the form of a mainframe, server, or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic or optical disk may carry the programming, and can be read by a suitable disk reader communicating with each processor at its corresponding station. Reference to a singular item, includes the possibility that there are plural of the same items present. “May” means optionally. Methods recited herein may be carried out in any order of the recited events which is logically possible, as well as the recited order of events.

“Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. By one item being “remote” from another, is referenced that the two items are at least in different buildings, and may be at least one mile, ten miles, or at least one hundred miles apart. An array “package” may be the array plus only a substrate on which the array is deposited, although the package may include other features (such as a housing). A “chamber” references an enclosed volume (although a chamber may be accessible through one or more ports). It will also be appreciated that throughout the present application, that words such as “top”, “upper”, and “lower” are used in a relative sense only. “Fluid” is used herein to reference a liquid. “Venting” or “vent” includes the outward flow of a gas or liquid. Reference to a singular item, includes the possibility that there are plural of the same items present. All patents and other cited references are incorporated into this application by reference.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used such as described in U.S. Pat. No. 5,599,695, U.S. Pat. No. 5,753,788, and U.S. Pat. No. 6,329,143. Interfeature areas need not be present particularly when the arrays are made by photolithographic methods as described in those patents.

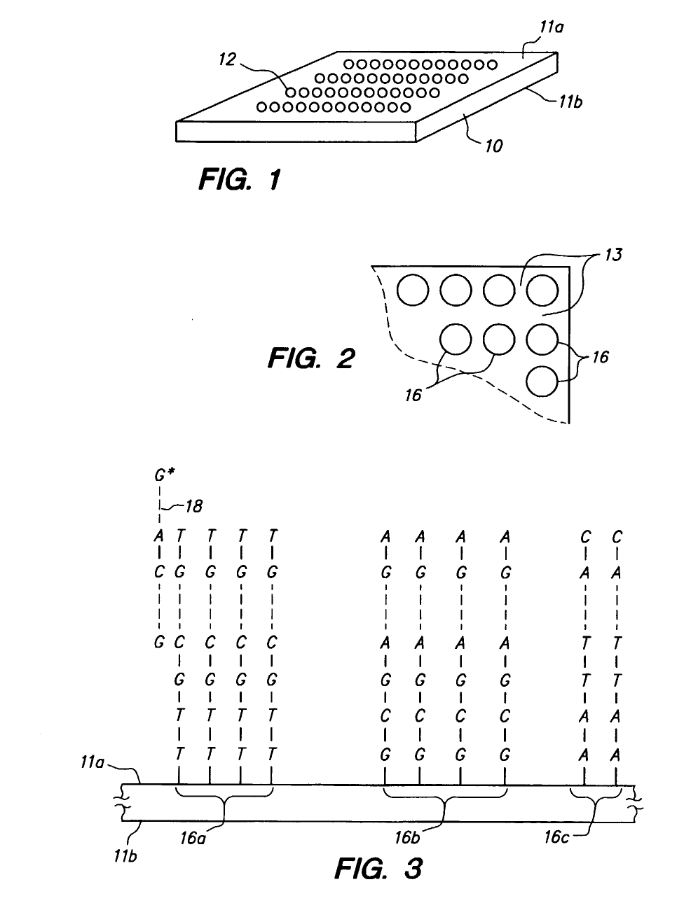

Referring first to FIGS. 1-3, a contiguous planar transparent substrate 10 carries multiple features 16 disposed across a first surface 11a of substrate 10 and separated by areas 13. Features 16 are disposed in a pattern which defines the array. A second surface 11b of substrate 10 does not carry any features. Substrate 10 may be of any shape although the remainder of the package of the present invention may need to be adapted accordingly. A typical array may contain at least ten features 16, at least 100 features, at least 1000 features, at least 100,000 features, or more. All of the features 16 may be different, or some could be the same as already described. Each feature carries a predetermined moiety or mixture of moieties which in the case of FIGS. 1-3 is a polynucleotide having a particular sequence. This is illustrated schematically in FIG. 3 where regions 16 are shown as carrying different polynucleotide sequences. Arrays of FIGS. 1-3 can be manufactured by in situ or deposition methods as discussed above. In use, a feature can detect a polynucleotide of a complementary sequence by hybridizing to it, such as polynucleotide 18 being detected by feature 16a in FIG. 3 (the “*” on polynucleotide 18 indicating a label such as a fluorescent label). Use of arrays to detect particular moieties in a sample (such as target sequences) are well known.

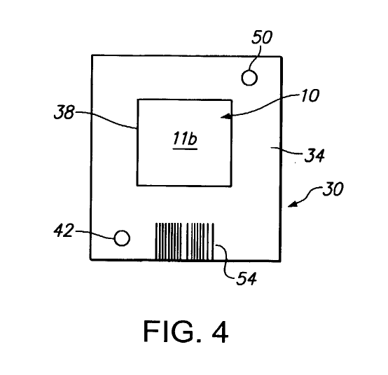

Referring now to FIG. 4 an array package 30 includes a housing 34 which has received substrate 10 adjacent an opening. Substrate 10 is sealed (such as by the use of a suitable adhesive) to housing 34 around a margin 38 with the second surface 11b facing outward. Housing 34 is configured such that housing 34 and substrate 10, define a chamber into which features 16 of array 12 face. This chamber is accessible through resilient septa 42, 50 which define normally closed ports of the chamber. Array package 30 preferably includes an identification (“ID”) 54 of the array. The identification 54 may be in the form of a bar code or some other machine readable code applied during the manufacture of array package 30. Identification 54 may itself contain instructions for a scanning apparatus that the interrogating light power for at least a first site of the sites to be scanned and of specified location on array package 30 should be altered (typically, decreased). These instructions are typically based on the expectation that the emitted signals from those sites will be too bright or that those sites are not of interest (for example, they are off the area covered by the array). The specified sites (specified by location on array package 30) can be particular ones of features 16 or can be other sites on array package 30 such as margin 38 from which, for example, unduly bright fluorescence from an adhesive might be expected, or regions off the area covered by the array and hence are not of interest (and hence the instructions describe the area to be scanned). Alternatively, identification 54 may be simply a unique series of characters which is also stored in a local or remote database in association with the foregoing location information. Such a database may be established by the array manufacturer and made accessible to the user (or provided to them as data on a portable storage-medium).

It will be appreciated though, that other array packages may be used. For example, the array package may consist only of the array of features 16 on substrate 10 (in which case ID 54 can be applied directly to substrate 10). Thus, an array package need not include any housing or closed chamber.

The components of the embodiments of the package 30 described above, may be made of any suitable material. For example, housing 34 can be made of metal or plastic such as polypropylene, polyethylene or acrylonitrile-butadiene-styrene (“ABS”). Substrate 10 may be of any suitable material, and is preferably sufficiently transparent to the wavelength of an interrogating and array emitted light, as to allow interrogation without removal from housing 34. Such transparent and non-transparent materials include, for flexible substrates: nylon, both modified and unmodified, nitrocellulose, polypropylene, and the like. For rigid substrates, specific materials of interest include: glass; fused silica, silicon, plastics (for example, polytetrafluoroethylene, polypropylene, polystyrene, polycarbonate, and blends thereof, and the like); metals (for example, gold, platinum, and the like). The first surface 11a of substrate 10 may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero or homopolymeric, and may or may not have separate functional moieties attached thereto (for example, conjugated), The materials from which substrate 10 and housing 34 (at least the portion facing toward the inside of chamber 36) may be fabricated should ideally themselves exhibit a low level of binding during hybridization or other events.

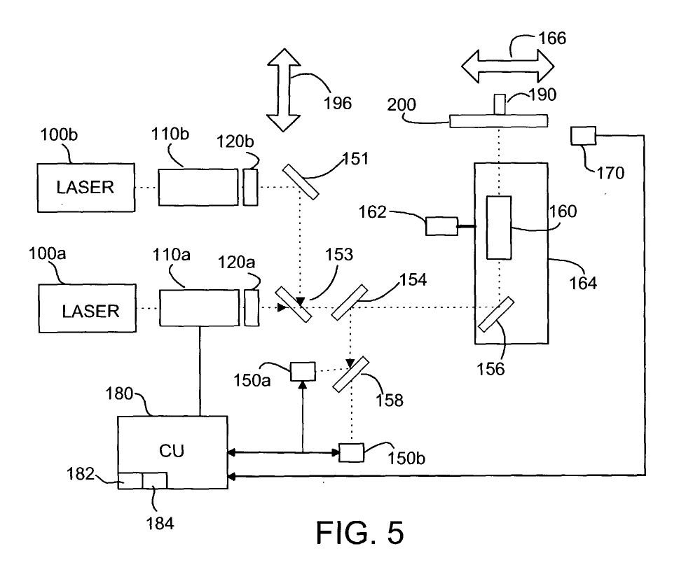

Referring now to FIG. 5, an apparatus of the present invention (which may be generally referenced as an array “scanner”) is illustrated. A light system provides light from a laser 100 which passes through an electro-optic modulator (EOM) 110 with attached polarizer 120. Each laser 100a, 100b may be of different wavelength (for example, one providing red red light and the other green) and each has its own corresponding EOM 110a, 110b and polarizer 120a, 120b. The beams may be combined along a path toward a holder 200 by the use of full mirror 151 and dichroic mirror 153. A control signal in the form of a variable voltage applied to each corresponding EOM 110a, 110b by the controller (CU) 180, changes the polarization of the exiting light which is thus more or less attenuated by the corresponding polarizer 120a, 120b. Controller 180 may be or include a suitably programmed processor. Thus, each EOM 110 and corresponding polarizer 120 together act as a variable optical attenuator which can alter the power of an interrogating light spot exiting from the attenuator in a manner, and for purposes, such as described in U.S. Pat. No. 6,406,849, the disclosure of which is herein incorporated by reference. The remainder of the light from both lasers 100a, 100b is transmitted through a dichroic beam splitter 154, reflected off fully reflecting mirror 156 and focused onto either an array 12 of an array package 30 mounted on holder 200, or a calibration member 230, whichever is at a reading position, using optical components in beam focuser 160. Light emitted, in particular fluorescence, at two different wavelengths (for example, green and red light) from features 16, in response to the interrogating light, is imaged using the same optics in focuser/scanner 160, and is reflected off mirrors 156 and 154. The two different wavelengths are separated by a further dichroic mirror 158 and are passed to respective detectors 150a and 150b. More optical components (not shown) may be used between the dichroic and each detector 150a, 150b (such as lenses, pinholes, filters, fibers etc.) and each detector 150a, 150b may be of various different types (e.g. a photomultiplier tube (PMT) or a CCD or an avalanche photodiode (APD)). All of the optical components through which light emitted from an array 12 or calibration member 230 in response to the illuminating laser light, passes to detectors 150a, 150b, together with those detectors, form a detection system. This detection system has a fixed focal plane.

A scan system causes the illuminating region in the form of a light spot from each laser 100a, 100b, and a detecting region of each detector 150a, 150b (which detecting region will form a pixel in the detected image), to be scanned across multiple regions of an array package 30 mounted on holder 200. The scanned regions for an array 12 will include at least the multiple features 16 of the array. In particular the scanning system is typically a line by line scanner, scanning the interrogating light in a line across an array 12 when at the reading position, in a direction of arrow 166, then moving (“transitioning”) the interrogating light in a direction into/out of the paper as viewed in FIG. 5 to a position at an end of a next line, and repeating the line scanning and transitioning until the entire array 12 has been scanned. This can be accomplished by providing a housing 164 containing mirror 158 and focuser 160, which housing 164 can be moved along a line of pixels (that is, from left to right or the reverse as viewed in FIG. 5) by a transporter 162. The second direction 192 of scanning (line transitioning) can be provided by second transporter which may include a motor and belt (not shown) to move holder 200 along one or more tracks. The second transporter may use a same or different actuator components to accomplish coarse (a larger number of lines) movement and finer movement (a smaller number of lines). The reader of FIG. 5 may further include a reader (not shown) which reads an identifier from an array package 30. When identifier 54 is in the form of a bar code, that reader may be a suitable bar code reader.

An autofocus detector 170 is also provided to sense any offset between different regions of array 12 when in the reading position, and a determined position of the focal plane of the detection system. An autofocus system includes detector 170, processor 180, and a motorized adjuster to move holder in the direction of arrow 196. A suitable chemical array autofocus system is described in pending U.S. patent application Ser. No. 09/415,184 for “Apparatus And Method For Autofocus” by Dorsel et al., filed Oct. 7, 1999, incorporated herein by reference, as well as European publication EP 1091229 published Apr. 11, 2001 under the same title and inventors.

Controller 180 of the apparatus is connected to receive signals from detectors 150a, 150b (these different signals being different “channels”), namely a signal which results at each of the multiple detected wavelengths from emitted light for each scanned region of array 12 when at the reading position mounted in holder 200. Controller 180 also receives the signal from autofocus offset detector 170, and provides the control signal to EOM 110, and controls the scan system. Controller 180 may also analyze, store, and/or output data relating to emitted signals received from detectors 150a, 150b in a known manner. Controller 180 may include a computer in the form of a programmable digital processor, and include a media reader 182 which can read a portable removable media (such as a magnetic or optical disk), and a communication module 184 which can communicate over a communication channel (such as a network, for example the internet or a telephone network) with a remote site (such as a database at which information relating to array package 30 may be stored in association with the identification 54). Controller 180 is suitably programmed to execute all of the steps required by it during operation of the apparatus, as discussed further below. Alternatively, controller 180 may be any hardware or hardware/software combination which can execute those steps.

A feature of controller 180 is that it is programmed to at least reduce the effect on scale factor resulting from control point adjustment made in response to laser degradation over time. In many embodiments, a feature of the controller 180 is that it is programmed to maintain a constant scale factor as the laser degrades over time and during use of the scanner, where the constant scale factor is maintained by modulation of both: (a) the interrogating power, e.g., through adjustment of the power attenuator (e.g., EOM 110); and (b) detector gain, e.g., through modulation of the detector itself (such as changing the voltage of a PMT) or through use of additional detector attenuation devices (such as filters, etc.). By “constant scale factor” is meant that the scale factor changes insubstantially between first and second temporal points, e.g., from a time before a change in control point to a time after a change in control point, where the magnitude of any change between the two relevant time points does not exceed about 50%, usually does not exceed about 10% and more usually does not exceed about 5% or 1%, if it is detectable at all. This feature of the of the controller 180 and of the invention is seen schematically in FIG. 5, where two-way arrows join the controller 180 to the detectors 150a and 150b. In certain embodiments, the controller is programmed to adjust the laser attenuator to maintain a constant interrogating power even as the output power of the laser decreases due to laser degradation. Upon reaching the control point or a margin limit relative to the control point where selection of a new control point is required in order to maintain control loop stability, the controller then decreases the power output of the laser, establishes a new control point and modulates, e.g., increases, the detector gain in a manner sufficient to maintain a constant scale factor, despite the decrease in power output and selection of new control point.

Basically, the detector gain increases to compensate for the decrease in laser power while maintaining a constant scale factor. Where desired, the controller 180 can make the above adjustment in interrogating power and detector gain separately and independently for all channels of the scanner. Where a single light source excites more than one chromophore in more than one channel, the controller may then adjust all detectors appropriately, e.g., equally, in order to maintain a constant scale factor in each channel. As such, the controller is programmed in scanner devices according to the present invention in a manner that maintains a constant scale factor despite a transition of laser output and control point from a first value to a second value, e.g., in response to laser degradation.

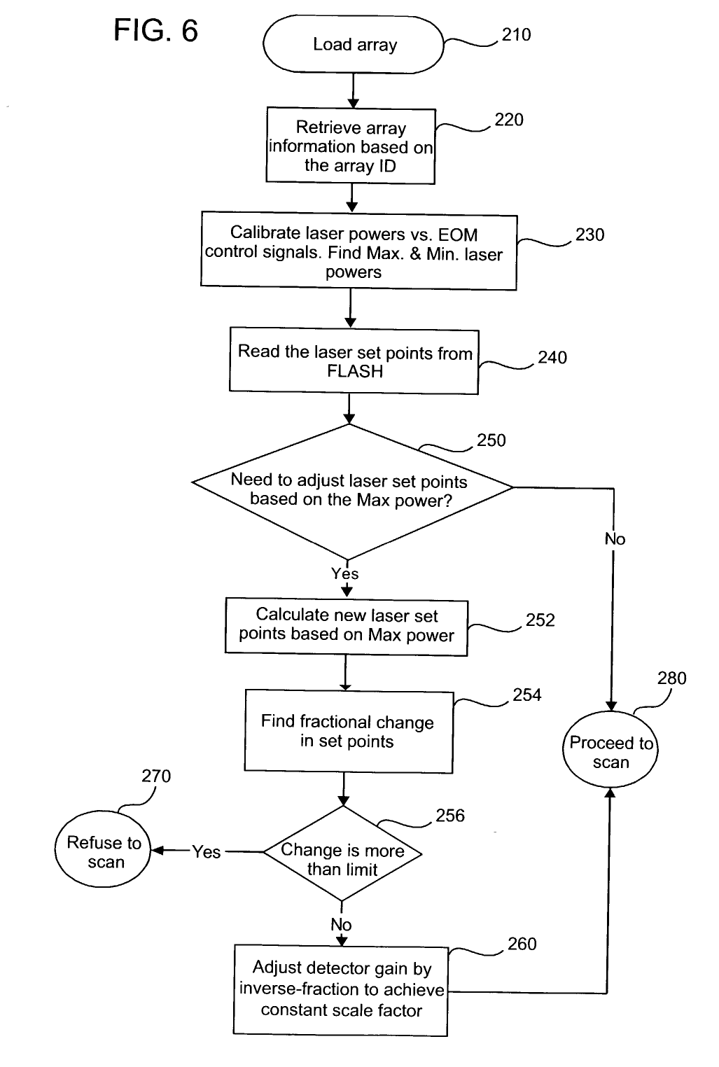

Operation of controller 180 according to the subject methods is further illustrated in FIG. 6. Following a given array package 30 being mounted in the apparatus as indicated by 210, the identifier reader may automatically (or upon operator command) read the array identifier (such as a bar code on the arrays substrate or housing) as indicated by step 220, and use this to retrieve information on the array layout (including characteristics of the array features, such as size, location, and composition). Such information may be retrieved directly from the contents of the read identifier when the read identifier contains such information. Alternatively, the read identifier may be used to retrieve such information from a database containing the identifier in association with such information. Such a database may be a local database accessible by controller 180 (such as may be contained in a portable storage medium in drive 182 which is associated with the array, such as by physical association in a same package with the array when received by the user, or by a suitable identification), or may be a remote database accessible by controller 180 through communication module 184 and a suitable communication channel (not shown). Next, the laser powers vs. EOM control signals are calibrated and the maximum and minimum laser powers are obtained, as indicated in step 230. Following this step, the laser set points are obtained, e.g., from the FLASH memory, as shown in step 240, and a determination (step 250) is made as to whether the laser set points need to be adjusted based on the maximum power output of the lasers. For example, when the difference in power of light from the light source and the power of interrogating light falls below a predetermined value or level, a determination may be made in step 250 to reset the laser points. In many embodiments, the “predetermined” value is set in software, for example as a value below which the control loop that maintains a constant interrogation light power from the EOM may become unstable because the interrogating light power is approaching the maximum laser source power. Following any desired recalculation of laser set points as shown in step 252, the fractional change in laser set points is determined, as shown in step 254. If the change is more than the limit, e.g., 1%, 2%, 5%, 10%, 20% or more, as desired, as determined in step 256, as decision may be made not to scan, as shown in step 270. A warning may also be provided to a user to replace the laser, since the difference in interrogating power and source power has fallen below a predetermined value. If the change is not more than the limit, the detector gain is adjusted to compensate for the decrease in interrogating factor and achieve the desired scale factor maintenance, as described above, where in certain embodiments the change in detector gain is the inverse fraction, as shown in step 260. For example, during use of a scanner according to one representative embodiment, as the laser power degrades and the maximum laser power is less than 1/0.85 times the control point, the controller sets the new control point as 0.85 times the laser maximum power. (It should be noted that the fraction of 0.85 of the laser power can be a replaced with different values and is only used as a reference to one embodiment.) Based on the resultant decrease in the control point to a fraction of the value it had before the laser power degraded to 1/0.85 of the set point, the controller then increases the detector gain by the inverse of that fraction such that the scale factor is held constant despite the decrease in laser output and selection of a new control point. Following detector gain adjustment, the array is then scanned as shown in step 280.

In practicing the subject methods, detector gain may be modulated using any convenient protocol, as indicated above. Where the detector is a PMT, while the relation between applied voltage and gain is nonlinear, the extent of change may be predicted utilizing the power law published by hardware vendors with empirically determined coefficients to make an estimate or by an iterative approach in testing gain obtained in varying voltage against expected results.

Using the subject methods to maintain scale factor in a scanner, scale factor may be maintained at a constant value over one or more changes in control point. In other words, the scale factor may be maintained at a constant value during a single change in the control point, or during several consecutive changes in the control point, thereby greatly extending the time that the scanner may be operated without having to adjust the scale factor. In scanners programmed according to the subject invention, the control point may be adjusted anywhere from 1 to 10 times, usually from 1 to 5 times, without causing the scanner scale factor to change. In particularly demanding situations, the change may be limited to 2 fold or 1.5 fold to limit the degradation of shot noise performance.

Controller 180 may also analyze, store, and/or output data relating to emitted signals received from detector 130 in a known manner. Controller 180 may include a computer in the form of a programmable digital processor, and include a media reader 182 which can read a portable removable media (such as a magnetic or optical disk), and a communication module 184 which can communicate over a communication channel (such as a network, for example the internet or a telephone network or a wireless channel) with a remote site (such as a database at which information relating to array package 30 may be stored in association with the identification 54). Controller 180 is suitably programmed to execute all of the steps required by it during operation of the apparatus, as discussed further below. Alternatively, controller 180 may be any hardware or hardware/software combination which can execute those steps.

The above-described scanning devices programmed as described above to maintain constant scale factor through interrogating power and detector gain modulation despite laser degradation and decrease in control point may be used in a number of assay protocols. One representative assay protocol is described in U.S. Pat. No. 6,406,849 the disclosure of which is herein incorporated by reference.

The interrogating light power is calibrated versus a control signal (step 230 in FIG. 6). Specifically, this is done by calibrating EOM 110 before the scan starts. In particular, the transmission of the EOM 110 is controlled using a high voltage differential input from controller 180. The power as a function of differential voltage is roughly sinusoidal with an offset from zero and scaling that varies with time and temperature. The maximum and minimum light powers and the corresponding control voltages are noted. Also the slope of the curve around the target light power (“set-point”) is measured. While scanning, on every scan row the light power is measured at a particular site or may be at the middle of the scan row. When the detected power is not equal to the predetermined target power, it signals EOM 110 so as to adjust the power to the target power by changing the control voltage to the EOM. Such a feedback control corrects for any drift in output power from laser 100 due to temperature or other fluctuations. All the checking and correction are preferably made during the relatively longer period of a transition from scanning one row to another (that is, the period where scanning features of one row has ended, until the period where scanning features of another row begins). This allows power fluctuations due to the corrections to be restricted to non-critical areas like scan turn-around period. Further, relatively little drift will likely occur during the scanning of a given row. In the present embodiment, only first order correction of interrogating light power is performed by converting a small power fluctuation into a linear power correction. The slope of the curve, calculated during initial calibration (230), is used to correct for the deviation in the light power from the target value. The next row is then scanned with any alterations in interrogating light power being executed as before. However, it will be appreciated that it is possible to adjust interrogating light power more frequently than just during the transition from one row to another (for example, when the scanning interrogating light spot is between features 16). Also in other embodiments it is possible to perform second or higher order corrections of the interrogating light power using the controller 180.

In certain embodiments it is not possible to control the light power at the maximum power. If the target power setting is at a local maximum and the output power drops, there is no way of telling which direction it went, and thus how to correct for it. Hence the target power should be less than the maximum light power. If the interrogating light power degrades resulting in a decrease in the maximum achievable light power, the set-point has to be adjusted accordingly. In the present embodiment if the target power is more than 85% of the maximum, the target power is modified to 85% of the maximum achievable power for the following scan (steps 250, 252 in FIG. 6.). Note that calibration (230) of EOM 110 before scanning each array corrects for any drifts in performance (for example due to temperature variations) between array scans. Further, use of an EOM 110 can allow for more rapid alteration of the interrogating light than may otherwise be possible by simply controlling power to some types of light sources, such as laser 100.

Note that a variety of geometries of the features 16 may be constructed other than the organized rows and columns of the array of FIGS. 1-3. For example, features 16 can be arranged in a series of curvilinear rows across the substrate surface (for example, a series of concentric circles or semi-circles of spots), and the like. Even irregular arrangements of features 16 can be used, at least when some means is provided such that during their use the locations of regions of particular characteristics can be determined (for example, a map of the regions is provided to the end user with the array). Furthermore, substrate 10 could carry more than one array 12, arranged in any desired configuration on substrate 10. While substrate 10 is planar and rectangular in form, other shapes could be used with housing 34 being adjusted accordingly. In many embodiments, substrate 10 will be shaped generally as a planar, rectangular solid, having a length in the range about 4 mm to 200 mm, usually about 4 mm to 150 mm, more usually about 4 mm to 125 mm; a width in the range about 4 mm to 200 mm, usually about 4 mm to 120 mm and more usually about 4 mm to 80 mm; and a thickness in the range about 0.01 mm to 5.0 mm, usually from about 0.1 mm to 2 mm and more usually from about 0.2 to 1 mm. However, larger substrates can be used. Less preferably, substrate 10 could have three-dimensional shape with irregularities in first surface 11a. In any event, the dimensions of housing 34 may be adjusted accordingly.

The apparatus of FIG. 5 can be constructed accordingly to scan array packages of the described structure.

As the scanner is employed, the controller of the system continually maintains the scale factor at a constant value by appropriately modulating the interrogating power and the detector gain, as described above. At some point during use of the scanner, e.g., when the control point is decreased from a first value to a second value because the laser output has fallen below an acceptable threshold level, in addition to appropriate modulation of detector gain, as described above, the programmed scanner may also alert the user that the laser is aged and that a replacement of the laser within a certain time frame is recommended. As the laser continues to degrade and successively lower control points are set, the detector gain can be further modulated according to the subject invention to maintain constant scale factor and increasingly stronger warnings can be generated. In addition, the scanner can be programmed to at some point quit maintaining a constant scale factor in order to further prompt the user to replace the laser.

The present invention provides for a number of distinct advantages over current approaches to maintaining a constant scale factor. One such advantage is that the user does not experience an abrupt reduction in scale factor when laser power falls below a control point, since modulation of detector gain according to the present invention maintains the scale factor at a constant value. As such, the present invention provides for a constant scale factor despite selection of successively lower control points. In addition, longer and more accurate warning times with respect to laser replacement may be obtained. Furthermore, the lifetime of a given laser prior to a reduction in scale factor is enhanced. Finally, the usable lifetime of a laser may be enhanced. As such, the subject invention represents a significant contribution to the art.

Obviously, the design disclosed in this patent application can be extended to the case of a non-linear relationship between fluorescence signal and control point (or laser power reaching the sample). For example, if calibration data are acquired and stored, these can be used later to compensate for such non-linear dependencies, e.g., due to saturation of the fluorescent dye label used.

As indicated above, the subject invention also provides programming designed to maintain constant scale factor during scanner use by modulating both interrogating power and detector gain. Programming according to the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present programming.

In addition to the representative scanner described above, the subject invention provides other biopolymer array optical scanners, which are programmed as described above. Any biopolymer optical scanner or device may be provided to include the above programming. Representative optical scanners of interest include those described in U.S. Pat. Nos. 5,585,639; 5,760,951; 5,763,870; 6,084,991; 6,222,664; 6,284,465; 6,329,196; 6,371,370 and 6,406,849—the disclosures of which are herein incorporated by reference.

The subject biopolymer optical scanners find use in a variety applications, where such applications are generally analyte detection applications in which the presence of a particular analyte in a given sample is detected at least qualitatively, if not quantitatively. Protocols for carrying out array assays are well known to those of skill in the art and need not be described in great detail here. Generally, the sample suspected of comprising the analyte of interest is contacted with an array under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system such as a fluorescent label present on the analyte, etc, where detection includes scanning with an optical scanner according to the present invention. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. References describing methods of using arrays in various applications include U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992—the disclosures of which are herein incorporated by reference.

Where the arrays are arrays of polypeptide binding agents, e.g., protein arrays, specific applications of interest include analyte detection/proteomics applications, including those described in U.S. Pat. Nos. 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

In using an array in connection with a programmed scanner according to the present invention, the array will typically be exposed to a sample (such as a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array.

Results from reading an array may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing). Stated otherwise, in certain variations, the subject methods may include a step of transmitting data from at least one of the detecting and deriving steps, to a remote location. The data may be transmitted to the remote location for further evaluation and/or use. Any convenient telecommunications means may be employed for transmitting the data, e.g., facsimile, modem, internet, etc.

Also provided are kits for use in connection with the subject invention. Such kits preferably include at least a computer readable medium including programming as discussed above and instructions. The instructions may include installation or setup directions. The instructions may include directions for use of the invention with options or combinations of options as described above. In certain embodiments, the instructions include both types of information.

Providing the software and instructions as a kit may serve a number of purposes. The combination may be packaged and purchased as a means of upgrading an existing scanner. Alternately, the combination may be provided in connection with a new scanner in which the software is preloaded on the same. In which case, the instructions will serve as a reference manual (or a part thereof) and the computer readable medium as a backup copy to the preloaded utility.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or world wide web. Some form of access security or identification protocol may be used to limit access to those entitled to use the subject invention. As with the instructions, the means for obtaining the instructions and/or programming is generally recorded on a suitable recording medium.

Various modifications to the particular embodiments described above are, of course, possible. Accordingly, the present invention is not limited to the particular embodiments described in detail above.

What is claimed is:

1. A method of reducing the effect on scale factor during use of an instrument for reading a biopolymer array when a control point of said instrument is adjusted from a first value to a second value, said method comprising:
(a) adjusting said control point from said first value to said second value; and
(b) adjusting detector gain of a detector of said instrument in a manner sufficient to reduce an effect on scale factor resulting from said adjustment.

2. The method according to claim 1, wherein said method is a method to maintain a constant scale factor.

3. The method according to claim 1, wherein said method further comprises modulating the power of interrogating light of said instrument prior to said adjustment (a) in order to maintain a constant scale factor.

4. A method according to claim 3, wherein the adjustments of (a) and (b) are performed when a difference in the power of light from the source and the interrogating power falls below a predetermined value.

5. A method according to claim 4, wherein a warning is provided to a user to replace the laser when said difference falls below a predetermined value.

6. A method according to claim 3, wherein said power of said interrogating light is modulated by adjusting an optical attenuator through which light from a light source must pass to provide said interrogating light.

7. The method according to claim 1, wherein said detector gain is increased in order to maintain said constant scale factor.

8. The method according to claim 1, wherein said detector gain is adjusted by modulating a detector.

9. The method according to claim 1, wherein said detector gain is adjusted by modulating an attenuator for a detector.

10. A computer-readable medium comprising a program that maintains a constant scale factor in an instrument for reading a biopolymer array by a method according to claim 1.

11. An instrument for reading biopolymer array programmed to maintain a constant scale factor by a method according to claim 1.

12. A method of assaying a sample, said method comprising:
(a) contacting said sample with a biopolymeric array of two or more biopolymer ligands immobilized on a surface of a solid support; and
(b) reading said array with an instrument for reading a biopolymer array according to claim 8 to obtain a result.

13. The method of claim 12, wherein said biopolymer array is chosen from a polypeptide array and a nucleic acid array.

14. The method of claim 12, further comprising transmitting said result from a first location to a second location.

15. The method of claim 14, where said second location is a remote location.

16. A method comprising receiving data representing said result of a scan obtained by the method of claim 12.

17. A kit for use in an instrument for reading a biopolymer array, said kit comprising:
(a) a computer-readable medium comprising programming that maintains a constant scale factor in a biopolymer array optical scanner by a method according to claim 1; and
(b) instructions for operating said instrument scanner according to said programming.
